(19) United States
(12) Patent Application Publication
(10) Pub. No.: US 2022/0378482 A1
(43) Pub. Date: Dec. 1, 2022

(54) ORTHOPEDIC BONE TAP AND METHODS INCORPORATING THE SAME

(71) Applicant: Spinal Generations, LLC, Mokena, IL (US)

(72) Inventors: Patrick Sweeney, Mokena, IL (US); Matthew V. Leyden, St. Paul, MN (US)

(21) Appl. No.: 17/804,011

(22) Filed: May 25, 2022

Related U.S. Application Data
(60) Provisional application No. 63/192,749, filed on May 25, 2021.

(51) Int. Cl. A61B 17/72; A61B 17/86

(52) U.S. Cl. CPC A61B 17/7225; A61B 17/86

(57) ABSTRACT

An orthopedic bone tap is provided, including a rod comprising a proximal end, a medial portion, and a distal end. The orthopedic bone tap also includes a plurality of cutting threads on the distal end of the rod. The orthopedic bone tap also includes an axial compression member engaged with at least one of the proximal end and the medial portion of the rod. The axial compression member is adapted and configured to exert compression that draws a bone fragment engaged with the plurality of cutting threads distally and against another bone fragment.

### CROSS-REFERENCE TO RELATED APPLICATION

This application claims the benefit of priority under 35 U.S.C. § 119(e) of U.S. Provisional Patent Application Ser. No. 63/192,749, filed May 25, 2021. The entire content of this application is hereby incorporated by reference herein.

### FIELD OF INVENTION

The present invention is directed to bone taps used in orthopedic procedures and, in particular, bone taps used in procedures involving unstable fractures such as intertrochanteric fractures of the hip and other skeletal structures.

### BACKGROUND

Proximal femoral fractures are serious injuries that often require surgery to repair. While the type of surgery depends on the location and severity of the fracture, the alignment of the broken bones, and the age and underlying health conditions of the patient, internal nails and screws are frequently used at the site of the fracture. Fixation in bone by implantation of orthopedic devices is often improved by tapping threads into a drilled bore. This can increase the fixation of the implanted threaded device. Fixation of fractured bone, a common use for these devices, may be composed of bone fragments that are separated in space as well as being misaligned in comparison to their natural pre-injury condition. The fragments may undergo fracture reduction by the surgeon to achieve better alignment and bony opposition but remain unstable and separate prior to fixation. Fracture compression is currently achieved after placing of fracture implants which are then used to obtain compression, often with a compression device. Typically, after completion of a surgery, a fractured bone will proceed to dynamically compress further into a stable condition, which can cause the implanted devices to protrude. In the case of an unstable intertrochanteric fracture, the implanted lag screw or similar device, can protrude laterally as the fracture compresses, causing a painful prominence on the side of the patient's upper thigh, or (in a worse situation) can penetrate through the femoral head into the pelvis. A unique way of addressing this common problem is to compress the fracture prior to placement of the fixation device, thus creating a more stable condition and allowing the selection of a more appropriately measured device which will be less likely to result in these painful conditions.

### SUMMARY

One aspect of the invention provides an orthopedic bone tap, including a rod comprising a proximal end, a medial portion, and a distal end. The orthopedic bone tap also includes a plurality of cutting threads on the distal end of the rod. The orthopedic bone tap also includes an axial compression member engaged with at least one of the proximal end and the medial portion of the rod. The axial compression member is adapted and configured to exert compression that draws a bone fragment engaged with the plurality of cutting threads distally and against another bone fragment.

Another aspect of the invention provides a method of using an orthopedic bone tap. The method includes the steps of: (a) drilling a channel in a fractured bone having a distal fragment and a proximal fragment; (b) advancing the orthopedic bone tap distally through the channel across the proximal fragment into the distal fragment; (c) rotating the orthopedic bone tap to cut threads within the distal fragment; and (d) actuating an axial compression member to induce a compressive force across the fractured bone in a controlled manner.

### BRIEF DESCRIPTION OF THE DRAWINGS

For a fuller understanding of the nature and desired objects of the present invention, reference is made to the following detailed description taken in conjunction with the accompanying drawing figures wherein like reference characters (i.e., the same reference character, or a reference character beginning with a “6” or “2” instead of a “1”) denote corresponding parts throughout the several views.

FIG. 1 illustrates a side view of an orthopedic bone tap in accordance with an exemplary embodiment of the invention;

FIG. 2 illustrates a side view of an orthopedic bone tap in an unstressed state in accordance with another exemplary embodiment of the invention;

FIG. 3 illustrates a side view of an orthopedic bone tap in a stressed state in accordance with another exemplary embodiment of the invention;

FIG. 4 illustrates a side view of a rotated axial compression member of an orthopedic bone tap in a stressed state in accordance with another exemplary embodiment of the invention;

FIG. 5 illustrates an unobstructed side view of an orthopedic bone tap in accordance with yet another exemplary embodiment of the invention;

FIGS. 6A-6B illustrate a perspective view of an orthopedic bone tap used in connection with an alignment device in accordance with various exemplary embodiments of the invention;

FIG. 7 is a flow diagram of a method of using an orthopedic bone tap in accordance with an exemplary embodiment of the invention.

### DEFINITIONS

The instant invention is most clearly understood with reference to the following definitions.

As used herein, the singular form “a,” “an,” and “the” include plural references unless the context clearly dictates otherwise.

As used in the specification and claims, the terms “comprises,” “comprising,” “containing,” “having,” and the like can have the meaning ascribed to them in U.S. patent law and can mean “includes,” “including,” and the like.

As used herein, the term “lateral” refers to a component that is located relatively away from the midline of a subject's body.

As used herein, the term “medial” refers to a component that is located between the proximal distal ends of a device.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Unless specifically stated or obvious from context, the term “or,” as used herein, is understood to be inclusive.

### DETAILED DESCRIPTION

The present invention relates to an orthopedic bone tap (sometimes referred to herein as an “orthopedic bone tap system” or a “bone tap”). Bone taps used for tapping pre-drilled bores in bone can provide additional utility through enabling compression of fractured bone fragments prior to final placement of an implant (e.g., a bone stabilizing implant). A compression device (e.g., an axial compression member) incorporated into the bone tap enables a more stable configuration of fractured bone to be achieved (as compared to conventional bone taps) and a more accurate bone screw length to be chosen prior to implant placement. Implanted screws may protrude into soft tissue causing pain as the fracture compresses. Earlier fracture compression can minimize the likelihood of this painful implant prominence and improve chances of procedure success.

Aspects of the present invention relate to an orthopedic bone tap that enables compression of bone fragments while using the bone tap rather than after placement of implants. Aspects of the present invention are directed primarily towards a threaded tapping device. Other aspects may be applied to other orthopedic instruments currently used prior to implant placement including a ratcheting accessory (e.g., a ratcheting axial compression member) that can apply force and/or stress (e.g., tensile, compressive, etc.) through the bone tap.

A bone tap is often used after a drill bores a channel through the interior of a bone or bone fragments. This use of a bone tap allows placement of threaded fixation implants in a controlled manner and can improve the strength of fixation. This procedure may be performed over a guidewire as a cannulated tap or in a less controlled manner using a solid tap. Bone taps exert torque on the interior of a bone to create a thread for an implant to be tightened into. Occasionally, the drilled channel and subsequent tapping action may be placed into foreign biologic materials or allograft already placed into a bony cavity or space. Regardless of the material being drilled and tapped, the purposes of the tapping action are to guide the implant placement and improve the implant fixation. In some situations, bone fragments may be so unstable as to require temporary stabilizing pins or screws prior to drilling or tapping to prevent rotation of the unstable fragment.

Until now, there has been no orthopedic bone tap enabling bone fragment compression prior to implant placement. Aspects of the present invention improve the utility of bone taps by enabling compression of the fractured fragments using the bone tap to exert a compression force (e.g., with an integral axial compression member, and accessory axial compression member, etc.).

In one embodiment of the present invention, threads of the bone tap are positioned at the end of a smooth shaft, in such a way that they terminate at the end (e.g., a distal end) of a drilled bone channel while being entirely across the fractured space, in such a way that the bone tap's threads rest only in the distal or final bone fragment and not in a proximal bone being fixed to. A threaded accessory may be attached to a proximal shaft of the bone tap. When such a threaded accessory is screwed down the shaft of the bone tap, the threaded accessory abuts the end of the firmly fixed guide tube (which may rest against the exterior surface of the bone). Consequently, this screwing movement draws the bone tap backwards (e.g., along an axis of the bone tap) through the guide tube, causing the fracture fragments to compress together. Once a desired fracture compression has been achieved, gradation markings (e.g., measurement gradations) on a surface of the bone tap may be used to determine an appropriate length of an implant with the bone in a more stable and compressed configuration.

One embodiment of the present invention applies a ratcheting accessory (e.g., an axial compression member) to a bone tap. The ratcheting accessory may be included or removed at certain times and provide controlled compression of the fracture through force exerted on the bone tap. Aspects of the present invention can be applied in different parts of the body with different fracture fixation devices.

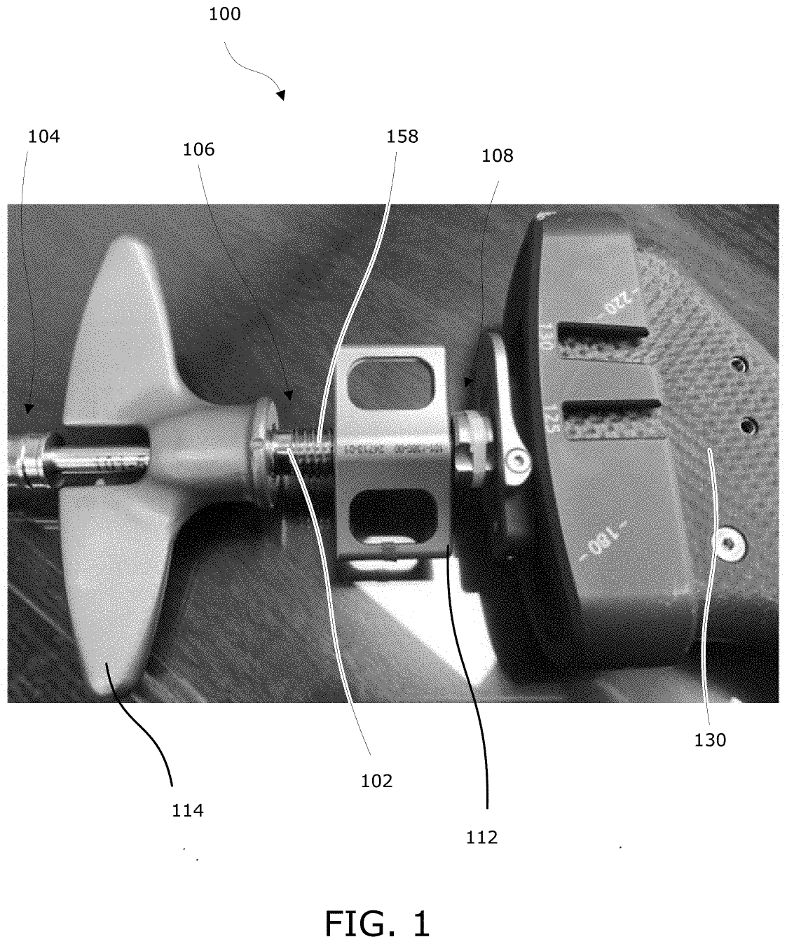

Referring now to FIG. 1, an orthopedic bone tap 100 is provided. Orthopedic bone tap 100 includes a rod 102 comprising a proximal end 104, a medial portion 106, and a distal end 108 (not visible). Rod 102 includes a plurality of threads 158 along the length of rod 102. Rod 102 may be either solid or cannulated. Orthopedic bone tap 100 also includes a plurality of cutting threads (not illustrated) on distal end 108 of rod 102. Orthopedic bone tap 100 is illustrated including a torque-application member 114. Torque-application member 114 is configured to impart a torque on rod 102 such that the cutting threads on a distal end of rod 102 can tap into a drilled bore (e.g., a “channel”) of a bone fragment. In some applications, a user can manually rotate torque-application member 114 to tap a bored bone fragment.

Orthopedic bone tap 100 also includes an axial compression member 112 engaged with medial portion 106 of rod 102. Axial compression member 112 is adapted and configured to exert compression on a bone fragment (e.g., by pressing against a guide 130). Axial compression member 112 draws bone fragment (e.g., by engaging the plurality of threads 158 against another bone fragment (not illustrated).

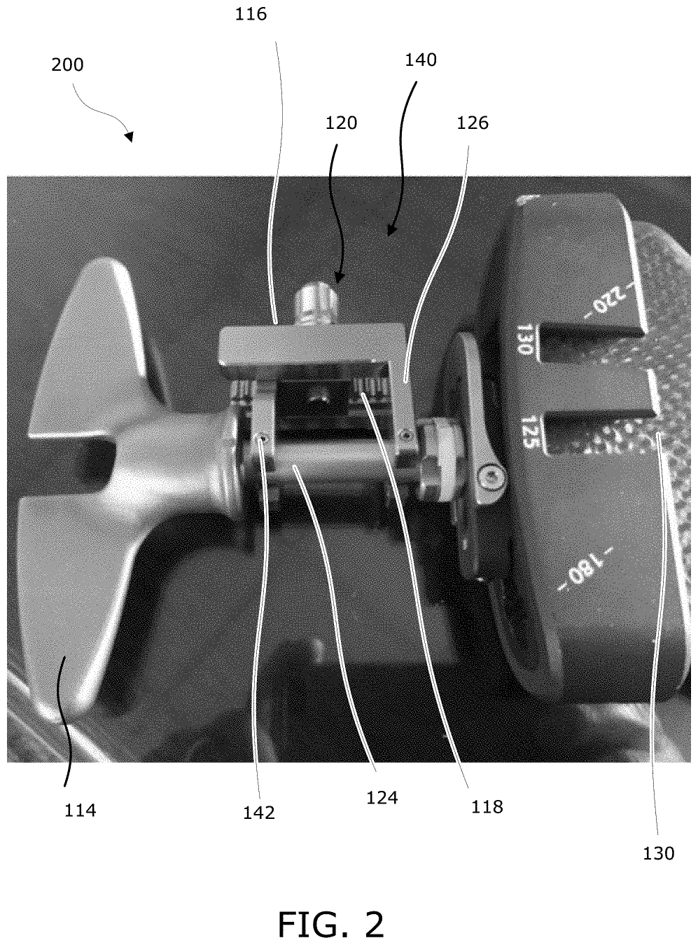

Referring now to FIG. 2, an orthopedic bone tap 200 is provided. Orthopedic bone tap 200 is similar to orthopedic bone tap 100 of FIG. 1 in many ways. Orthopedic bone tap 200 includes an axial compression member 116 (in lieu of axial compression member 112) and a rod 124 (in lieu of rod 102). Axial compression member 116 includes a rack-and-pinion system 140, including a rack 118, a pinion 120, and a plurality of links 126. Rack-and-pinion system 140 is configured to cause axial compression member 116 to translate along an axis of rod 124 (e.g., an axis along the length of rod 124). Axial compression member 116 is illustrated in an unstressed state (e.g., a state where bone fragment is not compressed). In other words, the illustrated configuration of axial compression member 116 does not substantially compress bone fragment against, for example, another bone fragment (not illustrated).

Rod 124 is a rod with a “smooth” (e.g., without threads) medial portion and/or proximal portion. While rod 102 interacts with axial compression member 112 through threads 158, rod 124 may use fastening structures 142 (e.g., hardware) to interact with axial compression member 116. Rod 124 (like rod 102) may be either solid or cannulated.

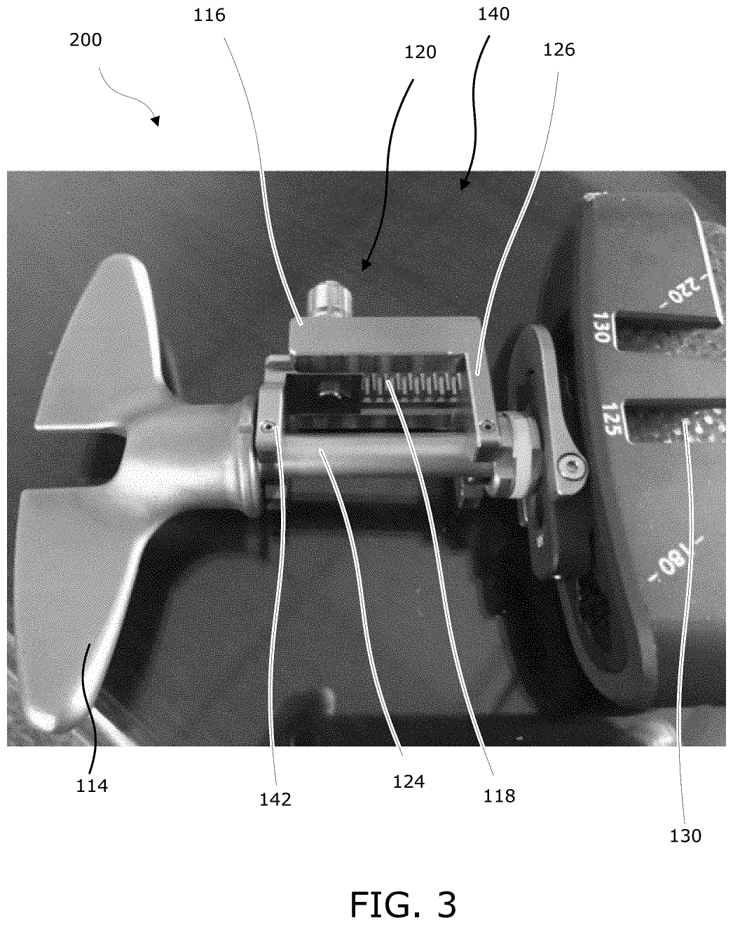

Referring now to FIG. 3, orthopedic bone tap 200 is illustrated in a stressed state (e.g., a state where bone fragment is compressed). Axial compression member 116 (including rack-and-pinion system 140) has been elongated by rotating pinion 120 engaged with rack 118 and/or by rotating axial compression member around rod 124. Consequently, links 126 induce stress into rod 124 (e.g., through fastening structures 142) such that the threads of rod 124, engaged with a bone fragment (not depicted), put the bone fragment into compression (e.g., with another bone fragment). Fastening structure 142, which is used to connect links 126 to rod 124, may include a number of structures, such as a screw, a bolt, a pin, a rivet, a threaded rod, or other hardware known in the art.

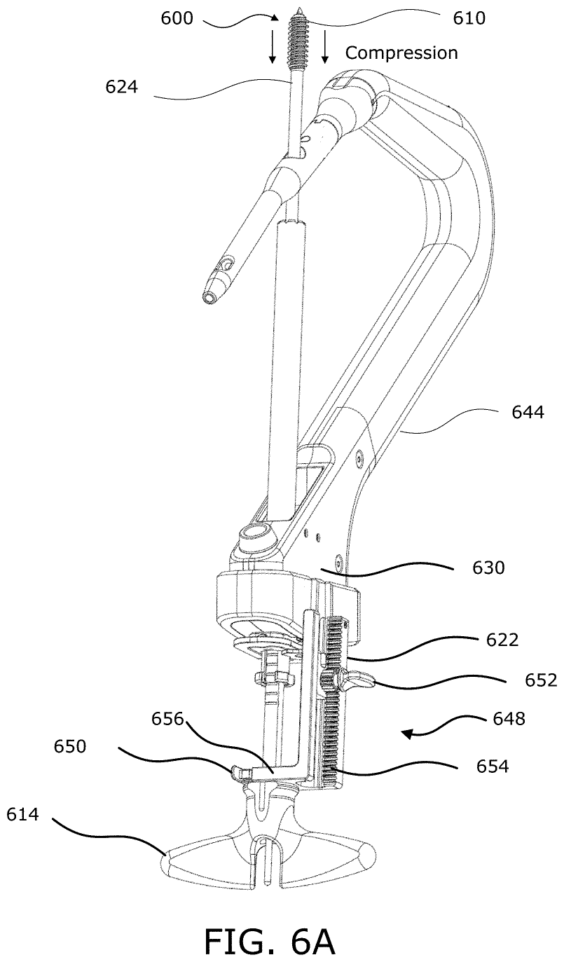

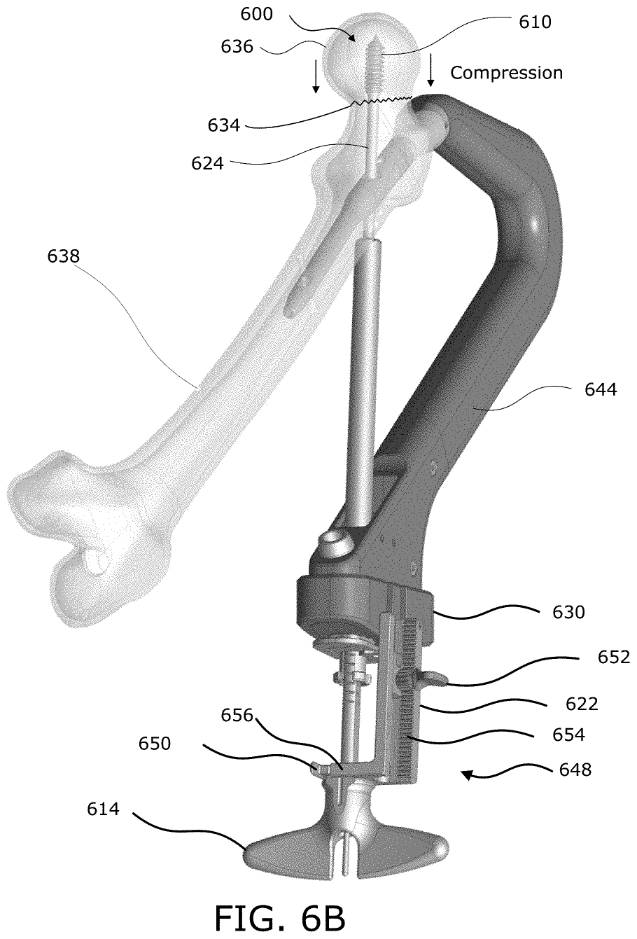

Although axial compression member 116 is illustrated including a rack-and-pinion system 140, the invention is not so limited. Another axial compression member may be used to induce stress (e.g., compression, tension, etc.) into bone fragment. For example, in one embodiment, a different rack-and-pinion system may be used (see axial compression member 622 of FIGS. 6A-6B). In another embodiment, another axial compression member may comprise a ratcheting device configured to engage with rod 124; the ratcheting device may be adapted and configured to control compression. In another embodiment, the axial compression member may include a collar, a threaded fastener (e.g., a nut), or another element to induce a stress (e.g., tensile stress, compressive stress, etc.) in the rod and/or a compressed state in a bone fragment. Relatedly, although only one bone fragment is described, the invention is not so limited. Another bone fragment or a plurality of bone fragments may be used in connection with the invention (e.g., see FIG. 6).

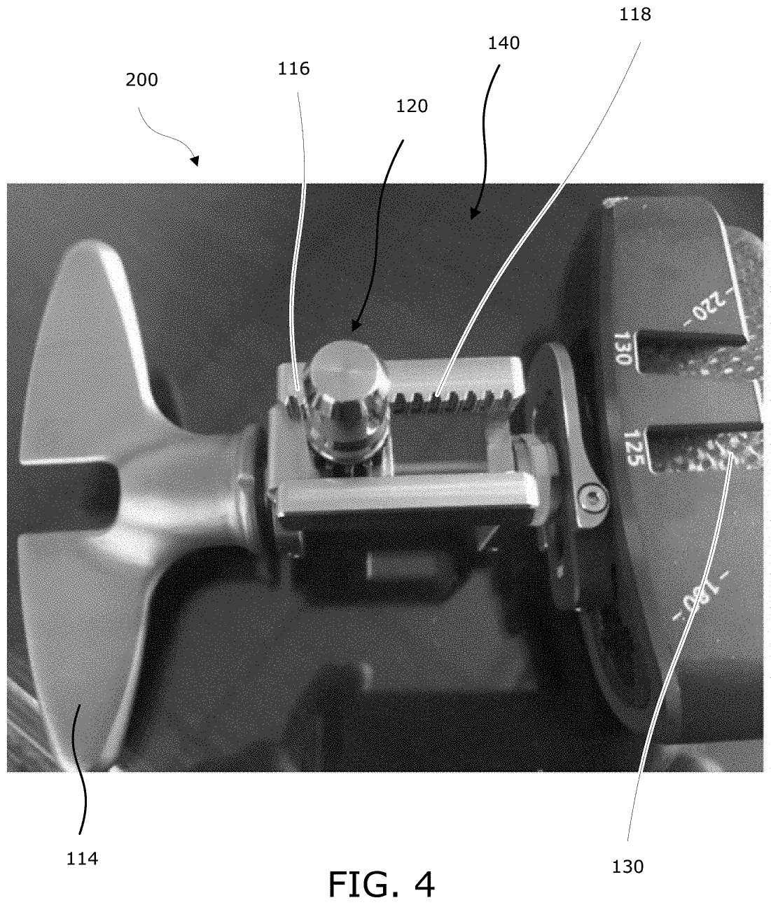

Referring now to FIG. 4, orthopedic bone tap 200 is illustrated in a top perspective view. Orthopedic bone tap 200 is in a similar configuration as the configuration illustrated in FIG. 3, except axial compression member 116 has been rotated approximately 90 degrees. As illustrated in FIG. 4, axial compression member 116 of orthopedic bone tap 200 may be rotated around an axis of rod 124 (e.g., the axis running along the length of rod 124).

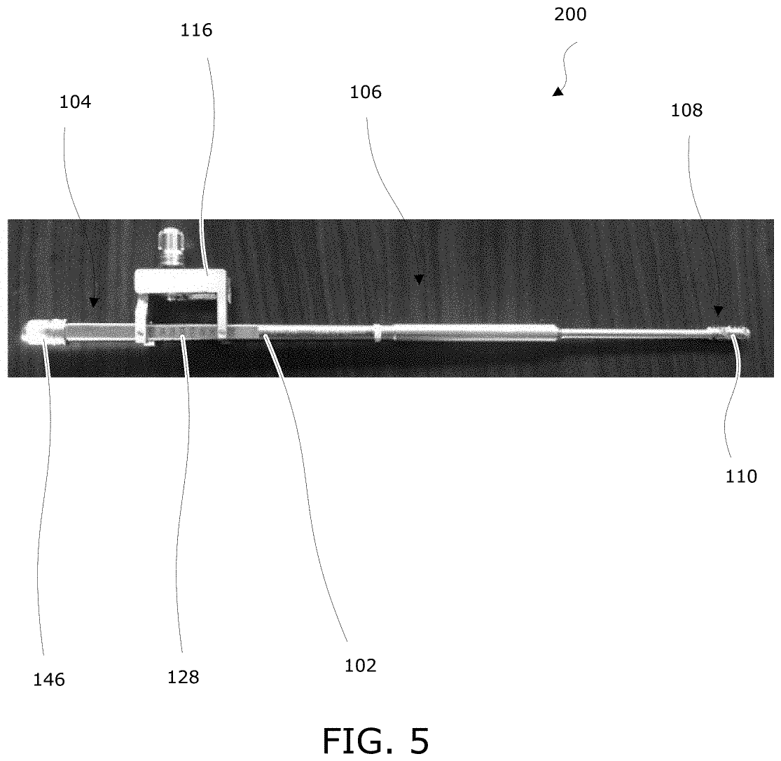

Referring now to FIG. 5, various sections of orthopedic bone tap 200 are more clearly illustrated. For example, proximal end 104, medial portion 106, and distal end 108 of rod 102 are illustrated. Medial portion 106 is illustrated as a smooth medial portion. A plurality of cutting threads 110 are depicted on distal end 108 of rod 102. Rod 102 also includes a plurality of measurement gradations 128. The plurality of measurement gradations 128 on rod 102 allow choosing an appropriate fracture implant based on a compressed configuration of a bone fragment. For example, the length of a channel through a fractured bone may be measured by using measurement gradations 128 on orthopedic bone tap 200 after axial compression member 116 induces compression into the bone fragment. An attachment point 146 is illustrated, which attaches to a fixture to provide leverage (e.g., for when the bone tap applies compression to the bone fragment). Attachment point 146 may be configured to interact with a variety of mating features (e.g., a socket, a flat switch, a pin, etc.). For example, attachment feature 146 may fit into a socket (e.g., with grooves) of a fixture and interlock with the fixture using mating features (e.g., grooves) of attachment feature 146. In another example, attachment feature 146 may have a through hole and keying feature that interlocks with a pin and keying feature of the fixture.

Referring now to FIGS. 6A-6B, an orthopedic bone tap 600 is illustrated being used in connection with an alignment device 644. Orthopedic bone tap 600 is similar in many ways to orthopedic bone tap 100 (see FIG. 1) or orthopedic bone tap 200 (see FIGS. 2-5); orthopedic bone tap 100 or 200 may be used in connection with alignment device 644 in lieu of orthopedic bone tap 600, in accordance with various exemplary embodiments of the invention. Orthopedic bone tap 600 includes a rod 624 (including a distal end, a medial portion, and a proximal end) and a plurality of threads 610 (e.g., cutting threads) on the distal end of rod 624. Orthopedic bone tap 600 includes an axial compression member 622 (including a rack-and-pinion system 648), which is similar in some ways to axial compression member 116.

Alignment device 644 comprises at least one bore for aligning orthopedic bone tap 600 through a bone fragment 638 (e.g., a femur). Alignment device 644 includes a guide 630. Guide 630 is configured to interact with an axial compression member 622. In some embodiments, a sheath may be inserted into the parallel bores of the guide before placement of orthopedic equipment (e.g., a bone tap, a lag screw, etc.). In some embodiments, the sheath may act to hold orthopedic equipment (e.g., a bone tap, a lag screw, etc.) in a desired position during a procedure (e.g., placing screws through a femur, tapping a bone with a bone tap, etc.). Alignment device 644 is provided to enable better alignment of, among other things, the orthopedic bone tap (e.g., 100, 200, 600, etc.)

Referring specifically to FIG. 6B, orthopedic bone tap 600 is illustrated in use in treating a fracture 634 by putting bone fragment 636 in compression against bone fragment 638 (e.g., a femur). As described herein, orthopedic bone tap 600 is configured to compress a bone fragment 636 (e.g., in the direction illustrated by a pair of arrows). Compression may be applied with axial compression member 622 (e.g., using rack and pinion system 648). Specifically, a pinion 652 may be turned (e.g., manually) in connection with a rack 654 to put bone fragment 636 in a compressed state. Alternatively, pinion 652 may be turned in an opposite direction to put bone fragment 636 in an uncompressed (or less compressed) state. The force/stress induced from axial compression member 622 (in connection with guide 630) is transferred to rod 624 at least in part by a link 656 and an attachment structure 650 (e.g., a clamp) of axial compression member 622. Link 656 may also interact with a feature (e.g., an integral feature of rod 624, an attached feature to rod 624, etc.) of orthopedic bone tap 600 to transmit force/stress. For example, a side of link 656 is illustrated adjacent to, and interacting with, a torque-application member 614. By pressing against torque-application member 614, a tensile stress is induced into rod 624 and a compressive stress is induced into bone fragment 636. It should be noted that another axial compression member (e.g., axial compression member 112, axial compression member 116 etc.) may be used here, in accordance with various exemplary embodiments of the invention.

### Materials

The components described herein can be made of any biocompatible material known to a person of skill in the art. In some embodiments, one or more components are made from a biocompatible metal. Exemplary biocompatible metals include, but are not limited to, stainless steel, titanium, aluminum, titanium alloy, cobalt-chromium alloy, and combinations thereof. In another embodiment, one or more components are made from a biocompatible plastic. Exemplary biocompatible plastics include, but are not limited to, polyvinyl chloride (PVC), polyethylene, polycarbonate, polyether ether ketone (PEEK), polyetherimide (PEI), polypropylene, polysulfone, polyurethane, and combinations thereof.

### Method of Using an Orthopedic Bone Tap System

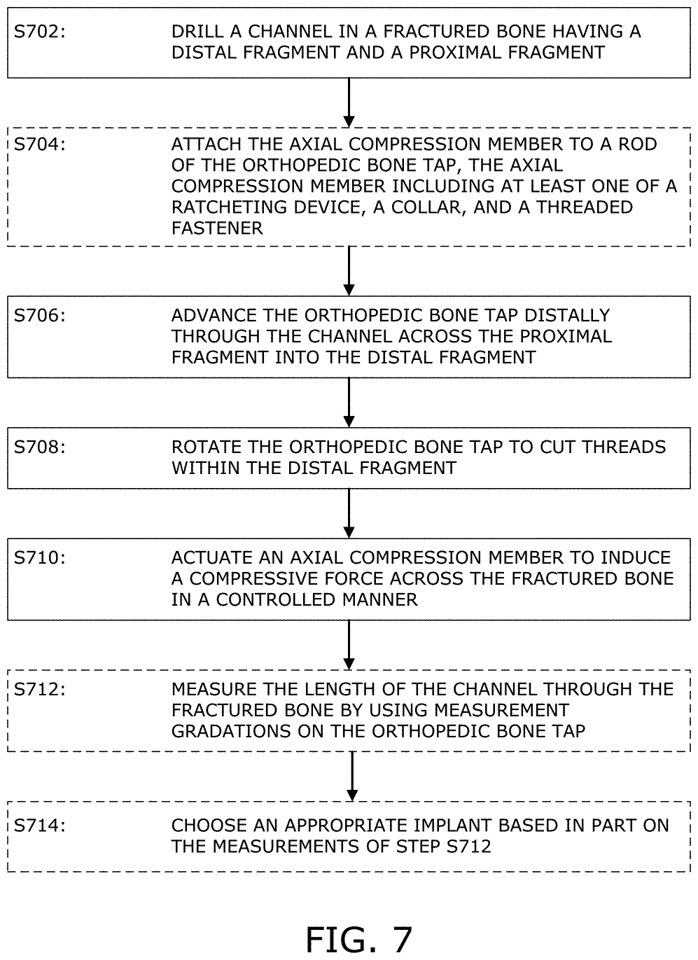

A flowchart depicting an exemplary method of using an orthopedic bone tap system (e.g., treating a proximal femoral fracture in a subject in need thereof) is illustrated in FIG. 7. It should be understood that the order of these steps can be rearranged within the scope of the invention, unless indicated otherwise. In step S702, a channel is drilled in a fractured bone having a distal fragment (e.g., bone fragment 636 of FIG. 6B) and a proximal fragment (e.g., bone fragment 638 of FIG. 6B). In optional S704, the axial compression member is attached to a rod of the orthopedic bone tap, the axial compression member including at least one of a ratcheting device, a collar, and a threaded fastener. It should be noted that optional step S704 may occur at any point prior to step s710. In step S706, the orthopedic bone tap (e.g., orthopedic bone tap 100, orthopedic bone tap 200, etc.) is advanced distally through the channel across the proximal fragment into the distal fragment. In step S708, the orthopedic bone tap is rotated (e.g., using torque application member 114) to cut threads within the distal fragment. In step S710, an axial compression member is actuated (e.g., by rotating axial compression member 112, by rotating pinion 120 of axial compression member 116, etc.) to induce a compressive force across the fractured bone in a controlled manner. In optional step S712, the length of the channel through the fractured bone is measured by using measurement gradations on the orthopedic bone tap. In optional S714, an appropriate implant is chosen based in part on the measurements of step S712.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. An orthopedic bone tap comprising:
a rod comprising a proximal end, a medial portion, and a distal end;
a plurality of cutting threads on the distal end of the rod; and
an axial compression member engaged with at least one of the proximal end and the medial portion of the rod, the axial compression member adapted and configured to exert compression that draws a bone fragment engaged with the plurality of cutting threads distally and against another bone fragment.

2. The orthopedic bone tap of claim 1 wherein the plurality of cutting threads are configured to tap into a drilled bore of the bone fragment.

3. The orthopedic bone tap of claim 1 further comprising:
measurement gradations on the rod to allow choosing an appropriate fracture implant based on a compressed configuration of the bone fragment.

4. The orthopedic bone tap of claim 1 wherein the medial portion is a smooth medial portion.

5. The orthopedic bone tap of claim 1 wherein the axial compression member comprises a ratcheting device configured to engage with the rod, the ratcheting device being adapted and configured to control compression.

6. The orthopedic bone tap of claim 1 wherein the axial compression member includes a rack-and-pinion system.

7. The orthopedic bone tap of claim 6 wherein the rack-and-pinion system is further configured to cause the axial compression member to translate along an axis of the rod.

8. The orthopedic bone tap of claim 1 wherein the axial compression member includes at least one of a collar and a threaded fastener.

9. The orthopedic bone tap of claim 1 wherein the rod is either solid or cannulated.

10. The orthopedic bone tap of claim 1 further comprising:
a torque-application member configured to rotate the rod to drive the plurality of cutting threads into the bone fragment.

11. A method of using an orthopedic bone tap, the method comprising:
(a) drilling a channel in a fractured bone having a distal fragment and a proximal fragment;
(b) advancing the orthopedic bone tap of claim 1 distally through the channel across the proximal fragment into the distal fragment;
(c) rotating the orthopedic bone tap to cut threads within the distal fragment; and
(d) actuating an axial compression member to induce a compressive force across the fractured bone in a controlled manner.

12. The method of claim 11 further comprising:
(e) measuring the length of the channel through the fractured bone by using measurement gradations on the orthopedic bone tap.

13. The method of claim 12 further comprising:
(f) choosing an appropriate implant based in part on the measurements of step (e).

14. The method of claim 11 further comprising:
(g) prior to step (d), attaching the axial compression member to a rod of the orthopedic bone tap, the axial compression member including at least one of a ratcheting device, a collar, and a threaded fastener.
